(19) United States
(12) Patent Application Publication
(10) Pub. No.: US 2026/0363643 A1
(43) Pub. Date: Nov. 19, 2026

(54) AI-aided whole-body Dual Energy X-ray Absorptiometry (DEXA or DXA) Imaging and Diagnostic Device

(71) Applicant: Yun Le, Cincinnati, OH (US)

(72) Inventor: Yun Le, Cincinnati, OH (US)

(21) Appl. No.: 19/206,557

(22) Filed: May 13, 2025

(51) Int. Cl. A61B 6/50; A61B 6/00; A61B 6/04; A61B 6/40; A61B 6/42; A61B 6/58

(52) U.S. Cl. CPC A61B 6/505; A61B 6/0407; A61B 6/4078; A61B 6/4208; A61B 6/4441; A61B 6/582

(57) ABSTRACT

The integration of dual cameras and application of AI systems and body thickness map within a dual-energy X-ray absorptiometry (DXA) imaging and diagnostic device marks an advancement in DXA technology. It enhances imaging and diagnostic processes by automating the selection of regions of interest for scanning, pre-processing images for edge detection, facilitating consistent body poses per patient for comparable results, and improving the accuracy of body composition imaging and analysis. This comprehensive approach signifies a progressive leap in DXA technology, improving workflow efficiency and diagnostic reliability in various healthcare settings.

### FIELD OF THE INVENTION

This invention relates to medical imaging and diagnostic devices, aimed at improving the accuracy and efficiency in detecting osteoporosis and analyzing body composition using Dual Energy X-ray Absorptiometry (DXA/DEXA) technology.

### BACKGROUND OF THE INVENTION

Dual Energy X-ray Absorptiometry (DXA/DEXA) is a standard technique for diagnosing osteoporosis, which is characterized by low bone mass. This method uses X-ray imaging at dual energy bands (low and high) to determine a patient's bone density. Bone density, or bone mineral density (BMD), measures the amount of bone mineral in specific areas such as the whole femur, femur neck, lumbar spine vertebrae, and forearm. Low bone density is associated with an increased risk of fractures and may indicate osteoporosis.

Osteoporosis commonly affects older individuals and increases the likelihood of fractures in the vertebrae, forearm bones, and hip bones. Fractures can result in reduced mobility and other complications, and severe cases, such as hip fractures, can be life-threatening. Diagnosing osteoporosis often requires radiographic techniques.

DXA works by transmitting a low energy band and a high energy band of X-ray through a targeted area of the patient's body and measuring their respective absorptions. The differential absorption of the low and high energy bands allows for the isolation of the bone's absorption, which is then used to estimate the patient's bone density.

The current DXA process necessitates manual intervention by the operator to specify scanning areas and analyze the resulting images. When multiple areas require scanning, the process involves manually repositioning the equipment and/or patient for each area. This procedure is inefficient and increases the potential for repetitive scans. Observations of the current equipment's usage indicate that operators often need to rescan the same area multiple times due to initial incorrect positioning. The manual area selection and scanning inaccuracies in the current process can expose patients to higher radioactive doses and negatively affect their experience.

To address these issues, the new DXA device includes overhead cameras, a body thickness measuring device, and artificial intelligence systems (AI agents) to assist or automate accurate selection of scan areas and to allow automatic scanning of multiple areas without manually repositioning the equipment or patient.

### SUMMARY OF THE INVENTION

The invention is a whole-body Dual Energy X-ray Absorptiometry (DXA/DEXA) imaging and diagnostic device with: 1) dual cameras mounted over the bed for live video feed and for whole body thickness estimation, 2) an AI-based body pose estimation system to determine key body points (left and right hip joints, lumbar spine, left and right forearm, etc.), 3) an AI-based object detection system to recognize and isolate key anatomical features (left and right femur, femur head, femur neck, greater trochanter, lumbar vertebrae, forearm, etc.), and 4) two line-laser lights indicating the center of X-ray and the two perpendicular scanning directions.

The cameras mounted over the bed allow the operator to visually observe the patient and the scanning process from a separate area or room. The body pose estimation and key point determination system automatically guide the scanning mechanism to target areas defined by the operator. The object detection recognizes and isolates anatomical features to automate and enhance DXA analysis. The whole-body thickness map obtained by the dual camera improves accuracy of scan area determination, bone density estimation, and body composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the subsequent detailed section of this description, the principles of the current application will be elaborated with reference to the example embodiments illustrated in the drawings, as follows:

FIG. 1—Orthographic Side View of the DXA Device

This screenshot offers an orthographic side view of the DXA device, including the stationary bed (101), the moving C-arm (102), and the dual cameras (103) mounted over the bed.

FIG. 2—Isometric Back View of the DXA Device

This drawing presents an isometric back view of the DXA device. The C-arm could move left and right.

FIG. 3—Internal Structure

This picture depicts the DXA device's internal structure, including the X-ray generator (106), X-ray detector (105), fan-beam X-ray (107), top tray (108) of the C-arm, and bottom tray (109) of the C-arm.

FIG. 4—Scanning Pattern

A scanning path that the machine follows is shown (110).

FIG. 5—A User Screen of the Operation Software

This is a screenshot of the operation software for the device, featuring the body pose estimation overlaid on the live video feed of a dummy patient, with the QR alignment codes highlighted by the software.

FIG. 6—Close View of the Line Lasers

Two line lasers (121,122) and their projected lines (123) are shown on this figure.

FIG. 7—X-ray Detector Sensor Opening

This figure shows two line lasers (121,122), dual camera (103), and the opening of the X-ray detector (124).

FIG. 8, 9, 10—AI-based Anatomical Feature Detection

FIG. 8 presents the training outcomes of an object detection AI agent designed to recognize the lumbar spine, vertebrae, femur, femur head, and greater trochanter. FIGS. 9 and 10 show actual results of recognized anatomical features.

### DETAILED DESCRIPTION OF THE INVENTION

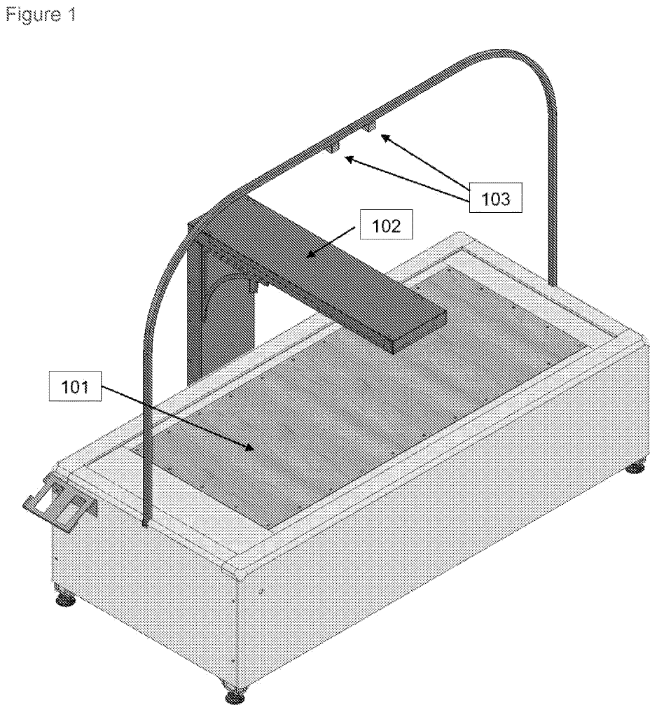

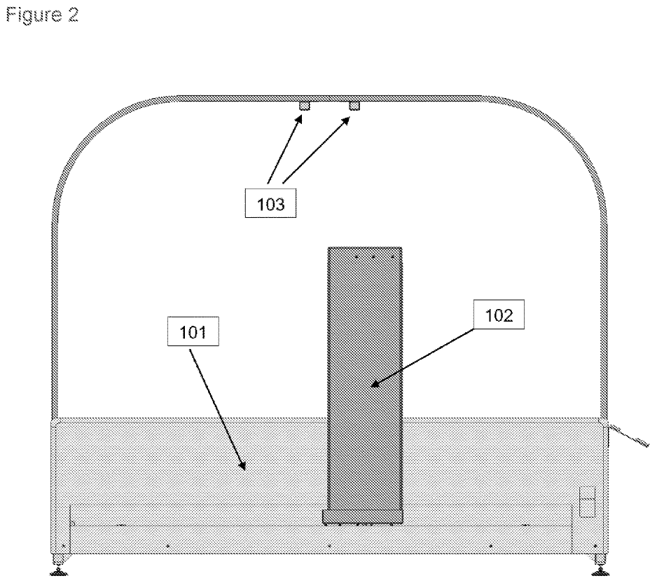

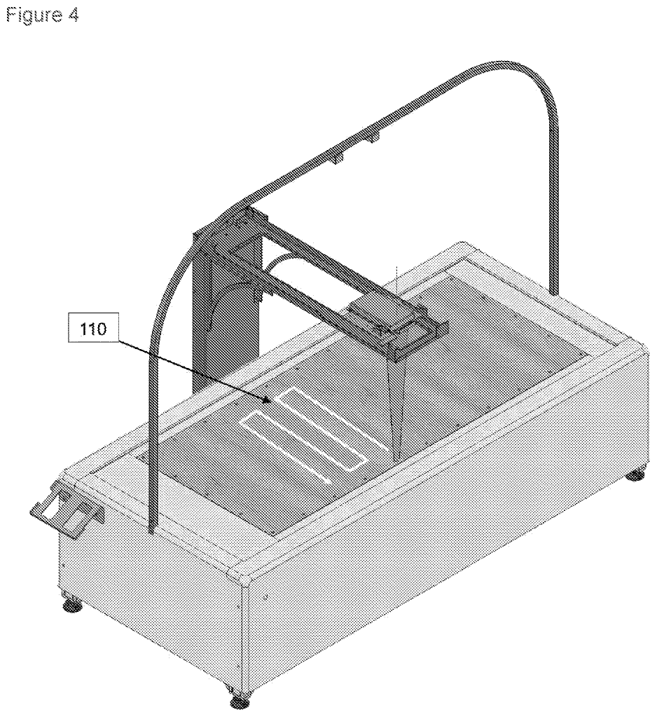

The DXA device, depicted in FIGS. 1, 2, and 3, comprises an X-ray generator (106), an X-ray detector (105), and a stationary bed (101) on which the patient lies. The C-arm (102), which houses the X-ray detector in its upper tray and the X-ray generator in its lower tray, rides on two parallel rails at the base of the bed. A driving mechanism within the C-arm synchronously moves the X-ray generator and detector along their respective lower (109) and upper (108) trays (referred to as the Width- or W-axis), ensuring that the detector remains aligned with the emitted fan-beam X-ray (107). Another mechanism drives the entire C-arm along the bed (referred to as the Length- or L-axis), perpendicular to the motion of the X-ray generator and detector. To generate an X-ray image of a region of interest (ROI) for a patient lying on the bed, the DXA device's software instructs these two driving mechanisms to first move the X-ray generator and detector to a corner of the ROI and then move along the pattern (110) as shown by FIG. 4. While moving along the W-axis, the software instructs the generator to emit an X-ray beam and the detector to collect X-ray photons. At the end of such a move, the software instructs the X-ray generator to stop emitting and the detector to stop collecting. This on-off scan across the W-axis is repeated along the L-axis until the entire ROI is covered. The above process is referred to as scanning or imaging an ROI.

Claim 1—A DXA Device with Dual Cameras

Two cameras are mounted directly above the bed, either on a frame affixed to the bed or on the ceiling of the room housing the DXA device. The distance between the two cameras may vary from a few centimeters to about twenty centimeters. The cameras are connected to the DXA device via USB or Ethernet. The use of cameras in the DXA device allows the operator to remotely define ROIs, control the device, and monitor the patient during the imaging process. It also enables additional functions such as automatically identifying anatomical features of interest (which become ROIs) for imaging through an AI-based body pose estimation system, measuring body thickness via a stereo computer vision algorithm, etc.

The integration of dual cameras in a dual-energy X-ray absorptiometry (DXA) device is both novel and functional, enhancing precision in patient positioning, optimizing scan quality, and improving diagnostic accuracy. Unlike conventional DXA systems, which rely solely on operator manually positioning at bed side for X-ray imaging, the use of dual cameras provides a unique, real-time visual reference at a location away from the bed side, facilitating better alignment and minimizing artifacts caused by improper positioning. As described and demonstrated in subsequent Claims, this innovation enhances workflow efficiency while ensuring more reliable bone density/body composition assessments, marking an advancement in DXA technology.

### Claim 2—QR Codes and Auto Alignment

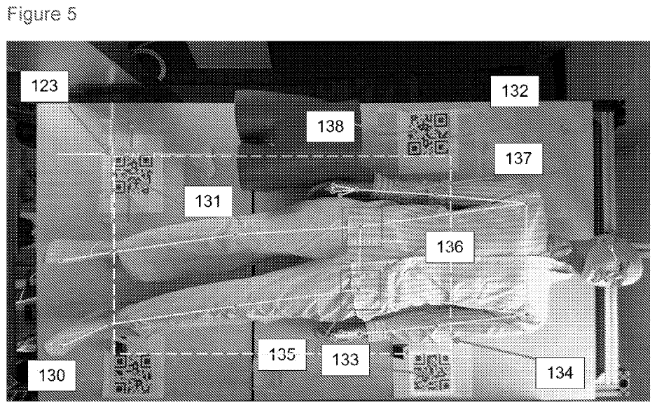

To define a Region of Interest (ROI) for imaging, either manually by an operator or automatically by an AI agent, the software must ascertain any physical point in 3D space from its corresponding camera point from the video feed in 2D space (and vice versa) at any given height between the bed and the detector. This process begins with establishing a bed-level mapping between the video feed and the corresponding physical coordinates along the L-axis and W-axis. To achieve this mapping, four sheets of paper with printed QR codes (as shown in FIG. 5) of letters LU (131), LD (130), RU (138), and RD (133) respectively are placed at the four corners (left-up (LU), left-down (LD), right-up (RU), and right-down (RD)) of the bed as reference points. The rectangle defined by the four vertices corresponding to the four locations of the QR codes LU, LD, RD, and RU is referred to as scan range (134) of the DXA device. The location of a QR code is determined by the coordinates of the corner that has a small square opposite to the corner without a square (132). The physical locations of these four QR codes are measured and stored in the DXA device. The letters of these codes are identified by the software using a QR code recognition system, and their camera coordinates are read and stored in the DXA device. The left-up corner of the bed's scan range corresponding to the location of the code LU is referred to as the origin. Physical coordinates at bed level are represented by motor steps along L-axis and W-axis. At origin, the physical coordinates are (0, 0). The right-down corner of the scan range has coordinates of (mRangeL, mRangeW), where mRangeL and mRangeW are the number of motor steps, along L-axis and W-axis respectively, needed to move from the origin to the right-down corner of the scan range. With both the physical and camera coordinates for each of the four corners acquired and stored, the software can accurately calculate the physical location (mL,mW) of any camera point (x,y) at bed level and vice versa using the sets of formulas provided below.

$$mL = mL UL + ( x - cLUL ) * \frac{mRangeL - mL UL}{cRDL - cLUL}$$

$$mW = mL UW + ( y - cLUW ) * \frac{mRangeW - mL UW}{cRDW - cLUW}$$

$$x = cLUL + ( mL - mL UL ) * \frac{cRDL - cLUL}{mRangeL - mL UL}$$

$$y = cLUW + ( mW - mL UW ) * \frac{cRDW - cLUW}{mRangeW - mLUW}$$

### Where:

(cLUL, cLUW) are camera coordinates of the left-up corner of the scan range.
(cRDL, cRDW) are camera coordinates of the right-down corner of the scan range.
(x, y) are coordinates along L-axis and W-axis of any given point in camera view.
(mL, mW) are motor steps along L-axis and W-axis to move from the origin (left-up corner identified by the QR code LU sheet placed at the left-up corner of the bed as described earlier) to the bed location corresponding to the point (x,y) in camera view.
(mLUL, mLUW) are the motor coordinates of the left-up corner of the bed.

### Claim 3—AI-Based Body Pose Estimation and Key Point Detection

The live video of one of the cameras is fed into an AI-based body pose estimation system. This pose estimation system is a real-time convolutional neural network that can recognize the camera coordinates of anatomical points (aka key points) of interest such as hip joints (135), shoulders, elbows, and wrists, as illustrated by FIG. 5.

These key points are 2D coordinates in camera view. To accurately define ROIs based on these 2D coordinates, the software will need depth information on these points. The following claims will specify how these 2D key camera points are mapped to physical points and then converted to regions of interest (ROIs) for scanning. Example ROIs are shown in ROI1 (136) and ROI2 in FIG. 5.

For each patient, the body pose information combined with the height obtained as described in Claim 4 is stored in the system for comparison during future visits. The bone densitometry result is sensitive to the patient's pose during imaging. Using historical body pose information, including height, the system would require a similar pose during scanning to produce more comparable results. It does this by comparing their current pose to a historic pose until it approximately matches, then communicates to the operator that the pose is ready to scan. This is later described in Claim 7.

### Claim 4—Body Thickness Map

When an operator or AI agent draws a region of interest (ROI) over a patient's body using the camera view, the ROI appears on the surface of the patient's body but does not have any depth information. To scan the correct area, the software requires the thickness of the body at the ROI location to calculate the coordinates of the ROI projected down to the bed level. To obtain the body thickness information (body thickness map) including at the ROI region, the software uses the following formula per pixel on the camera feed.

$$Thickness = H - \frac{f * B}{d}$$

### Where:

H is the average distance from the two cameras to the bed.
f is the focal length of the cameras.
B is the baseline distance between the two cameras.
d is the disparity, which is the distance between the object's positions in the left and right camera images.
   Claim 5—Determine Region for Scan from Camera View ROI

With the average body thickness across an ROI region, the software then uses the following formula to calculate the corresponding ROI at bed level for scanning.

$${BedROI}_{top} = ( 1 - hH ) * {ROI}_{top} + hH * {Y}_{cameraCenter}$$

$${BedROI}_{left} = ( 1 - hH ) * {ROI}_{left} + hH * {X}_{cameraCenter}$$

$${BedROI}_{width} = {ROI}_{width} + ( 1 - hH )$$

$${BedROI}_{height} = {ROI}_{height} + ( 1 - hH )$$

$$hH = \frac{ROIAboveBed}{CameraAboveBed}$$

### Where:

ROI is the region of interest drawn in camera view.
BedROI is the calculated ROI at bed level in camera view for scanning.
(ROI_{width}, ROI_{height}) are the dimensions of the ROI.
(ROI_{left}, ROIₜₒₚ) are the camera coordinates of the left-up corner of the ROI.
(BedROI_{width}, BedROI_{height}) are the dimensions of the BedROI.
(BedROI_{left}, BedROIₜₒₚ) are the camera coordinates of the left-up corner of the BedROI.
(X_{cameraCenter}, Y_{cameraCenter}) is the center point of the camera view in camera coordinates. For example, for camera with a resolution of 1280×720, then X_{cameraCenter} equals 640 and Y_{cameraCenter} equals 360.
ROIAboveBed is the distance between the ROI and the bed. It equals the average body thickness of the ROI region.
CameraAboveBed is the distance between the camera and the bed.

With the calculated ROI at bed level (BedROI) in camera view, the software then uses the equations in Claim 2 to determine its physical location (represented by motor coordinates (mROI_{left}, mROIₜₒₚ) of the left-up corner) and dimension (represented by motor steps (mBedROI_{width}, mBedROI_{height}) along the L-axis and W-axis). Finally, the software instructs the driving mechanism to move the X-ray generator and detector to (mROI_{left}, mROIₜₒₚ) and start scanning the range (mBedROI_{width}, mBedROI_{height}).

### Claim 6—Converting Key Points to ROIs

Using the body thickness info (Claim 4) at key points (Claim 3) and the mapping from camera to physical coordinates (Claim 2), the software could convert any interested key points into rectangular regions of interest (ROI) for the device to scan. For example, the following formula defines an ROI with some fixed width and height covering a detected key point.

$${x}_{ROI} = {x}_{keypoint} - \frac{{width}_{ROI}}{2}$$

$${y}_{ROI} = {y}_{keypoint} - \frac{{height}_{ROI}}{2}$$

### Where:

(x_{ROI}, y_{ROI}) is the left-up corner of ROI in camera view.
width_{ROI} is some fixed width of ROI adequate to cover the region of interest.
height_{ROI} is some fixed height of ROI adequate to cover the region of interest.

x_{keypoint} is the x coordinate of the AI detected key point in camera view
y_{keypoint} is the y coordinator of the AI detected key point in camera view

The software determines a region of interest (ROI) for the lumbar spine using four key points detected by AI from the left and right hips and shoulders, as specified by the following formula.

$${x}_{hip} = \frac{{x}_{leftHip} + {x}_{r ightHip}}{2}$$

$${x}_{shoulder} = \frac{{x}_{leftShoulder} + {x}_{rightShoulder}}{2}$$

$${x}_{LumbarROI} = {x}_{hip} + 0.3 * ( {x}_{shoulder} - {x}_{hip} )$$

$${y}_{LumbarROI} = \frac{{y}_{leftHip} + {y}_{r ightHip} + {y}_{leftShoulder} + {y}_{rightShoulder}}{4}$$

### Where:

(x_{leftHip}, y_{leftHip}) is the AI detected camera coordinates of patient's left hip joint.
(x_{rightHip}, y_{rightHip}) is the AI detected camera coordinates of patient's right hip joint.
(x_{leftShoulder}, y_{leftShoulder}) is the AI detected camera coordinates of patient's left shoulder joint.
(x_{rightShoulder}, y_{rightShoulder}) is the AI detected camera coordinates of patient's right shoulder joint.
(x_{lumbarROI}, y_{lumbarROI}) is the center of ROI for imaging lumbar spine. Its width and height could be predefined to cover the entire lumbar spine vertebrae of L1, L2, L3, and L4.

### Claim 7—Consistent Body Pose and Comparable Result

The body pose includes key points (Claim 3) and their height (Claim 4) above the bed, which are recorded in the system for each patient visit. During the preparation phase for scanning, the system continuously monitors and displays the patient's body pose and detected key points on the operator's screen (FIG. 5). It also compares the current body pose with historical data stored in the system. The operator then guides the patient to assume and maintain a pose similar to their previous visits. The system will communicate to the operator when a similar pose is achieved, at which point the scanning process can commence. The bone densitometry result is sensitive to the patient's pose during imaging. This procedure minimizes any inconsistencies arising from variations in body pose.

### Claim 8—AI-Based Anatomical Feature Recognition

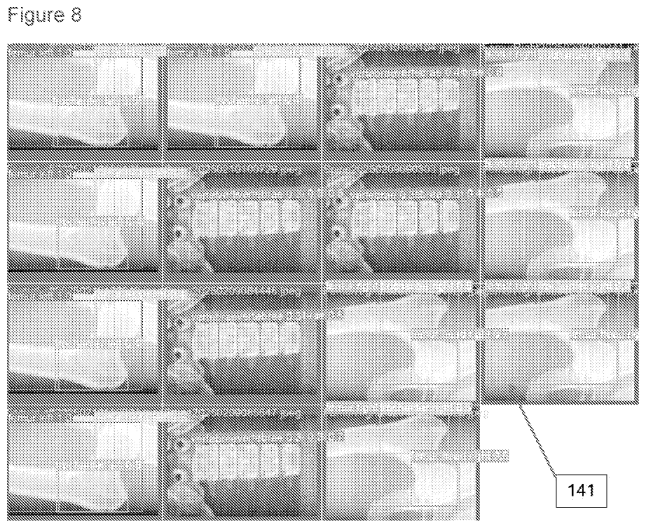

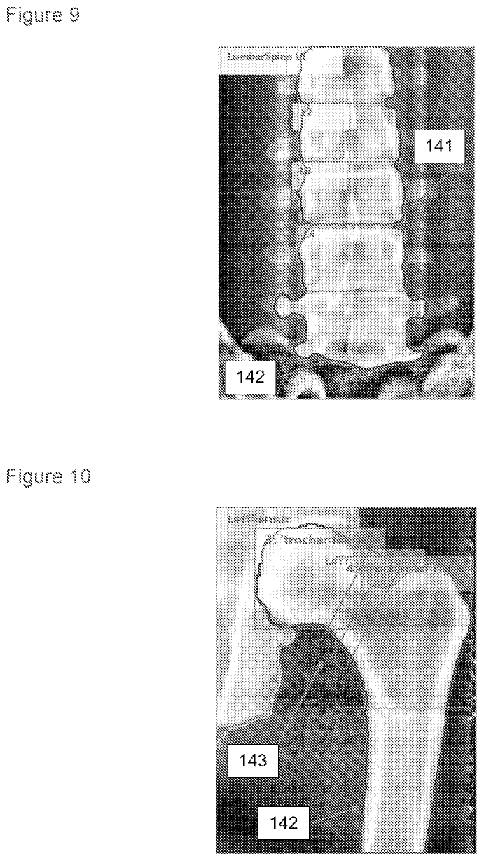

After scanning an ROI, the software applies an AI-based object detection algorithm to the scanned image to identify key anatomical features such as whole femur, femur head, great trochanter, whole lumbar, lumbar vertebrae, etc. Each identified anatomical feature is marked by a bounding rectangle box (141) as illustrated by FIG. 8.

Subsequently, the software utilizes bounding boxes to identify the edges of the femur, lumbar spine, and other significant anatomical features. By establishing these bounding boxes, the software focuses its edge detection analysis solely on the pixels within these areas, thereby significantly enhancing the accuracy and reliability of the process.

FIG. 9 shows the software's capabilities to automatically recognize the lumbar (141) and its vertebrae (141) and then detect its edge (142).

Furthermore, the software employs the bounding boxes for the femur head and the greater trochanter to calculate a rotated rectangular bounding box for the femur neck using the specified formula below.

$${x}_{rot atedRect} = \frac{{x}_{femurHead} + {x}_{greatTrochanter}}{2}$$

$${y}_{rot atedRect} = \frac{{y}_{femurHead} + {y}_{greatTrochanter}}{2}$$

$${Angle}_{rotatedRect} = {tan}^{- 1} ( \frac{{y}_{femurHead} - {y}_{greatTrochanter}}{{x}_{femurHead} - {x}_{greatTrochanter}} )$$

$${width}_{rotatedRect} = \frac{Abs ( {x}_{femurHead} - {x}_{greatTrochanter} )}{4}$$

$${height}_{rotatedRect} = Abs ( {y}_{femurHead} - {y}_{greatTrochanter} ) * 2$$

### Where:

(x_{femurHead}, y_{femurHead}) is the center of the femur head bounding box.
(x_{greatTrochanter}, y_{greatTrochanter}) is the center of the great trochanter bounding box.
(x_{rotatedRect}, y_{rotatedRect}) is the center of the femur neck bounding box (a rotated rectangle).
Angle_{rotatedRect} is the rotating angle of the femur head bounding box.
width_{rotatedRect} is the width of the rotated rectangle.
height_{rotatedRect} is the height of the rotated rectangle.

FIG. 10 illustrates the AI agent's capability to automatically detect the edge of the left femur (142, indicated by the blue line) and its neck (143, shown as a mid-upper rotated rectangle) following its automatic identification of the boundary (outer rectangular box), head (left upper box), and great trochanter (right box).

### Claim 9—Using Line Lasers

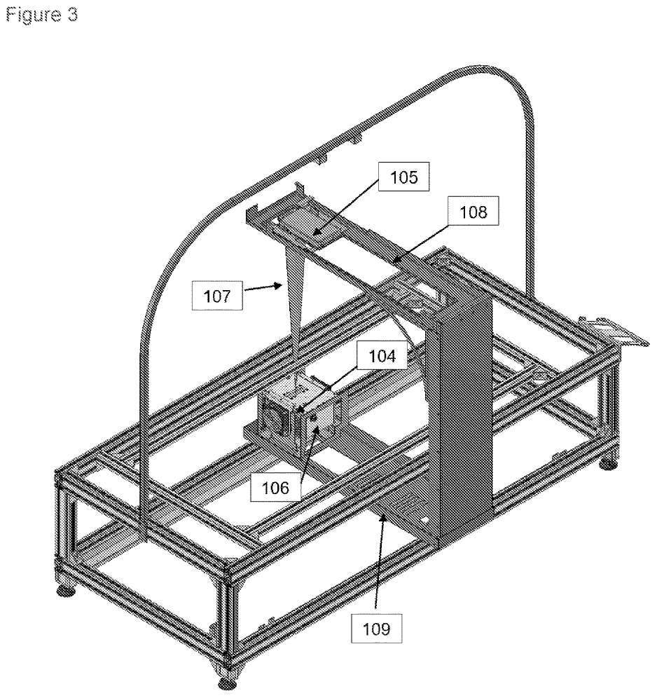

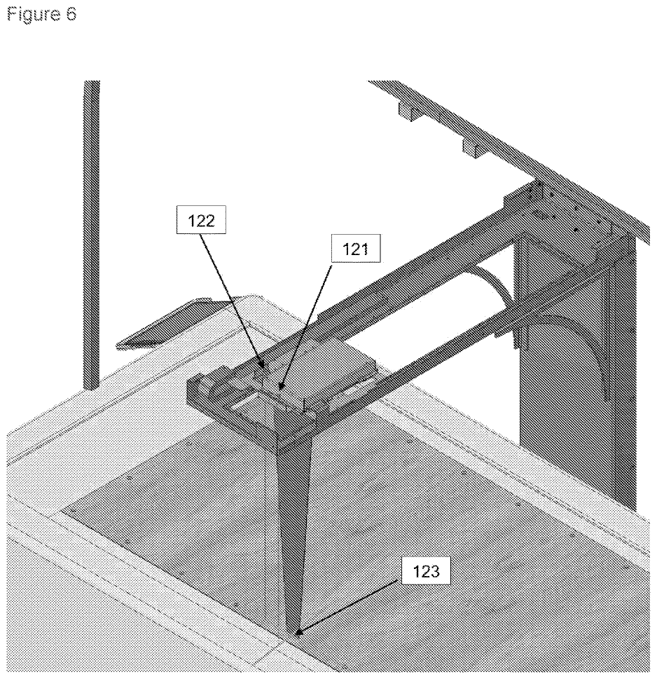

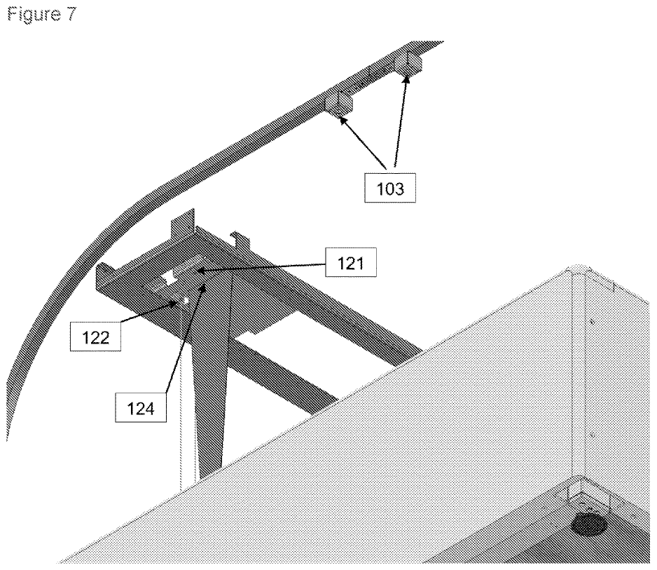

A pair of line lasers (121,122) is installed adjacent to the X-ray detector as illustrated in FIG. 6. These line lasers are projected onto the bed below, with their beams perpendicular to the bed and their projected lines on the bed perpendicular to each other (123). The intersection point (123) of the two laser lines on the bed, the focal point (104, FIG. 3) of the X-ray generator, and the center of the detector's photon collection array (124, FIG. 7) form a straight line, with the intersection point positioned above the focal point and below the detector center. The two laser lines projected onto the bed are parallel to the length and width of the bed respectively.

Using two line lasers has some advantages over the single dot laser approach used in most current devices. The intersection point of the two lines is precisely aligned with the X-ray generator and detector, allowing for accurate positioning of the device for imaging. During setup or maintenance, the engineer can use the two laser lines to align the X-ray generator and detector by first making the beams of the lasers perpendicular to the bed surface and then positioning the X-ray generator so that its focal point is at the intersection of the two projected laser lines.

### Claim 10—Using Body Thickness Map for Fat Mass Estimation

The body thickness map obtained through the method described in Claim 4 can enhance the reliability and accuracy of bone densitometry and fat mass estimation. Dual-energy radiography (DXA technology) enables the identification of two material constituents by measuring absorptions at low and high energy bands. To estimate body fat mass, it is necessary to identify three component materials: bone, fat, and other tissues. This requires additional data, such as body thickness, along with the absorptions at low and high energy bands, to solve the equations accurately.

The inventions claimed are:

1. A whole-body Dual Energy X-ray Absorptiometry (DXA) imaging and diagnostic device, comprising, (a) a stationary bed (101), (b) a fan-beam X-ray generator (106), (c) a multi-energy-band X-ray detector (105), (d) a moving C-arm (102) that houses the X-ray generator and detector on its bottom and top trays respectively, (e) a driving mechanism that synchronizes the movement of the X-ray generator and detector along the bottom tray (109) and top tray (108) of the C-arm to ensure continuous alignment of the detector with the X-ray beam, (f) dual cameras located above the bed and the C-arm (103), looking down at the patient lying on the bed.

2. A method according to claim 1, wherein the software automatically aligns the physical coordinates of the bed to the camera coordinates using QR codes placed at four corners of the bed (130, 131, 133, 138) and captured in the camera feed.

3. A method according to claim 1, wherein the camera feed is fed into an AI-based pose estimation system to identify anatomical points (key points) on the patient's body, including but not limited to the left and right hips, lumbar spine, left hand and forearm, and right hand and forearm.

4. A method according to claim 1, wherein the dual camera feeds are utilized by the software to generate a thickness map of the patient's body.

5. A method according to claims 2 and 4, wherein a region of interest (ROI) drawn in camera view is used to determine the physical region for scanning.

6. A method according to claims 2 and 3, wherein the key points identified by the pose estimation system are used to automatically determine regions of interest (ROIs) in camera view for scanning.

7. A method as described in claims 3 and 4, wherein the patient's body pose data, including key points and their heights, are recorded in the system to facilitate consistent poses in future scans of the same patient, thereby ensuring comparable results.

8. A method according to claim 1, wherein the resulting X-ray scans (images) are processed by an AI-based object detection system to identify key anatomical features in the image and narrow the image down for more efficient and reliable edge detection, and key anatomical features include, but are not limited to, the lumbar spine, vertebrae of the lumbar spine, left and right femurs, femur heads, greater trochanters, radius and ulna of forearms, and bones of the hands (phalanges, metacarpals, carpals).

9. A method according to claim 1, wherein a pair of line lasers is employed for accurate positioning during scanning and for facilitating alignment of the X-ray detector and generator and bed during setup and maintenance.

10. A method according to claim 4, wherein the body thickness map generated by the dual camera images is utilized to more accurately determine the body composition—including fatty tissue, muscle, and bone—of the patient.
